(19) United States
(12) Patent Application Publication
(10) Pub. No.: US 2011/0184418 A1
(43) Pub. Date: Jul. 28, 2011

(54) Unsealed, Non-Colloidal Radiopharmaceutical Compositions and Methods for Treatment of Abnormal Tissue

(76) Inventors: Les Wold, Rochester, MN (US); Ole Poulsen, Boca Raton, FL (US)

(21) Appl. No.: 12/883,325

(22) Filed: Sep. 16, 2010

Related U.S. Application Data
(60) Provisional application No. 61/243,713, filed on Sep. 18, 2009.

(51) Int. Cl. A61B 17/16; A61K 51/00; A61K 33/00; A61K 33/08; A61P 35/00

(52) U.S. Cl. 606/80; 424/1.11; 424/722; 424/692

(57) ABSTRACT

The present disclosure relates, according to some embodiments, to compositions and methods for treatment of an abnormal tissue (e.g., cancer). For example, a method may comprise administering to a subject in the region of the abnormal tissue a composition (e.g., a pharmaceutical and/or radiopharmaceutical composition) having a basic pH (e.g., greater than about 9). A radiopharmaceutical composition may comprise, in some embodiments, an admixture of (a) a first radionuclide composition comprising an unsealed, non-colloidal, radioactive, free holmium-166 (¹⁶⁶Ho) and/or unsealed, non-colloidal, radioactive, free metal ion of holmium-166 (¹⁶⁶Ho) (e.g., ¹⁶⁶Ho³⁺) and (b) a second radionuclide composition comprising an unsealed, non-colloidal, radioactive, free lutetium-177 (¹⁷⁷Lu) and/or an unsealed, non-colloidal, radioactive, free metal ion of lutetium-177 (¹⁷⁷Lu) (e.g., ¹⁷⁷Lu³⁺). A radiopharmaceutical composition may comprise, in some embodiments, an unsealed, non-colloidal, radioactive, free metal and/or metal ion (e.g., antimony-126, antimony-127, cesium-132, europium-136, gadolinium-159, lutetium-177, phosphorous-32, promethium-148, promethium-149, rubidium-86, strontium-89, tin-125, ytterbium-175, and/or ions thereof).

### CROSS-REFERENCE TO RELATED APPLICATIONS

This application claims the benefit of U.S. Provisional Application No. 61/243,713 filed Sep. 18, 2009, the entire contents of which are hereby incorporated in their entirety by reference.

### FIELD OF THE DISCLOSURE

The present disclosure relates, in some embodiments, to compositions and methods for treatment of an abnormal tissue.

### BACKGROUND OF THE DISCLOSURE

In 2006 the American Cancer Society (ACS) estimated that there would be approximately 1,400,000 new cases of cancer diagnosed. The ACS also estimated there would be approximately 565,000 deaths due to cancer in 2006. Although malignant melanoma may be considered an uncommon malignancy, approximately 160,000 cases are diagnosed annually worldwide. Once melanoma has developed distant metastasis, it is generally considered incurable.

Many anti-cancer agents are toxic to healthy cells and tissues. In addition, some anti-cancer agents have products (e.g., degradation products, decay products, or other downstream products) that are toxic to healthy cells and tissues. For example, alpha, beta, and gamma-emitting radionuclides may have one or more decay products (e.g., heavy metals) that may have an adverse or otherwise undesirable impact on healthy cells and tissues.

### SUMMARY

Accordingly, a need has arisen for improved methods and compositions for delivering an agent to a cancer cell and/or a cancer tumor to limit contact of the anti-cancer agent with healthy cells and tissues.

The present disclosure relates, according to some embodiments, to methods for locoregional treatment of abnormal tissue in a human subject. For example, a method may comprise administering to a subject in the region of the abnormal tissue a radiopharmaceutical composition (e.g., liquid) having a basic pH (e.g., greater than about 9). A radiopharmaceutical composition may comprise, in some embodiments, an admixture of (a) a first radionuclide composition comprising an unsealed, non-colloidal, radioactive, free holmium-166 (¹⁶⁶Ho) and/or unsealed, non-colloidal, radioactive, free metal ion of holmium-166 (¹⁶⁶Ho) (e.g., ¹⁶⁶Ho³⁺) and (b) a second radionuclide composition comprising an unsealed, non-colloidal, radioactive, free lutetium-177 (¹⁷⁷Lu) and/or an unsealed, non-colloidal, radioactive, free metal ion of lutetium-177 (¹⁷⁷Lu) (e.g., ¹⁷⁷Lu³⁺). According to some embodiments, an abnormal tissue may be selected from bone cancer, breast cancer, colon cancer, lung cancer, prostate cancer, skin cancer, and combinations thereof. An abnormal tissue may include, in some embodiments, metastatic disease.

According to some embodiments, a method for locoregional treatment of abnormal tissue in a subject (e.g., a human, a non-human animal) may comprise administering to a subject in the region of the abnormal tissue a radiopharmaceutical composition having a basic pH (e.g., greater than about 9) and comprising an unsealed, non-colloidal, radioactive, free metal and/or metal ion. For example, a radiopharmaceutical composition may comprise, in some embodiments, antimony-126 (¹²⁶Sb) antimony-127 (¹²⁷Sb), cesium-132 (¹³²Cs), europium-136 (¹³⁶Eu), gadolinium-159 (¹⁵⁹Gd), lutetium-177 (¹⁷⁷Lu), phosphorous-32 (³²P), promethium-148 (¹⁴⁸Pm), promethium-149 (¹⁴⁹Pm), rubidium-86 (⁸⁶Rb), strontium-89 (⁸⁹Sr), tin-125 (¹²⁵Sn), ytterbium-175 (¹⁷⁵Yb), and/or ions thereof.

A method for locoregional treatment of abnormal tissue may include administering, a volume of a radiopharmaceutical composition (e.g., comprising a single radionuclide or a combination of ¹⁶⁶Ho and ¹⁷⁷Lu) of less than about two microliters per cubic centimeter of abnormal tissue in some embodiments. A method for locoregional treatment of abnormal tissue may include, according to some embodiments, administering, a volume of a radiopharmaceutical composition of at least about two microliter per cubic centimeter of abnormal tissue. For example, the volume of the administered composition may be from about two microliters per cubic centimeter of abnormal tissue to about ten microliters per cubic centimeter of abnormal tissue. In some embodiments, a radiopharmaceutical composition may have a pH greater than about 10.0, greater than about 11.0, greater than about 12.0, and/or greater than about 14.0. A radiopharmaceutical composition may comprise a salt (e.g., a pharmaceutically acceptable saline), in some embodiments. A radiopharmaceutical composition may comprise, according to some embodiments, a base selected from sodium hydroxide, potassium hydroxide, barium hydroxide, strontium hydroxide, rubidium hydroxide, magnesium hydroxide, and combinations thereof.

According to some embodiments, a radiopharmaceutical composition may be administered by injection. For example, a method may comprise intratumorally injecting the radiopharmaceutical composition, intralesionally injecting the radiopharmaceutical composition, intramuscularly injecting the radiopharmaceutical composition, interstitially injecting the radiopharmaceutical composition, intrathecally injecting the radiopharmaceutical composition, intraosseously injecting the radiopharmaceutical composition, intraorbitally injecting the radiopharmaceutical composition, intraparenchymally injecting the radiopharmaceutical composition, or combinations thereof. Administering may include, according to some embodiments, injecting the radiopharmaceutical composition into or through a bone. For example, a method may comprise drilling a hole in the bone.

According to some embodiments, a majority of an injected radiopharmaceutical composition may linger at the injection site for a period of time sufficient to have a therapeutic effect. Various metrics for assessing localization to the injection site may be used as desired and/or required. For example, a majority (e.g., at least eighty percent, at least ninety percent, and/or at least ninety-five percent) of the total remaining (i.e., undecayed) radioactivity of (i) the first radionuclide composition or (ii) the second radionuclide composition may remain within one centimeter, two centimeters, and/or three centimeters of the injection site for at least two hours, at least four hours, at least eight hours, and/or at least twelve hours after the administering. A majority (e.g., at least eighty percent, at least ninety percent, and/or at least ninety-five percent) of the total remaining radioactivity of (i) the first radionuclide composition or (ii) the second radionuclide composition may remain within one centimeter, two centimeters, and/or three centimeters of the injection site for at least one half life of the radionuclide composition with the shorter half life after the administering. In some embodiments, one or more reciprocal metrics may be used. For example, less than twenty percent, less than ten percent, and/or less than five percent of the total remaining radioactivity of (i) the first radionuclide composition or (ii) the second radionuclide composition is located more than one centimeter away from the injection site within the first two hours, the first four hours, the first eight hours, and/or the first twelve hours after the administering. Less than twenty percent, less than ten percent, and/or less than five percent of the total remaining radioactivity of (i) the first radionuclide composition or (ii) the second radionuclide composition is located, according to some embodiments, more than one centimeter from the injection site within a time after the administering that is equivalent to one half life of the radioactive free metal ion with the shorter half life.

The present disclosure relates, according to some embodiments, to methods for locoregional treatment of abnormal tissue in a subject (e.g., a human, a non-human animal). For example, a method may comprise administering to a subject in the region of the abnormal tissue a pharmaceutical composition (e.g., liquid) having a basic pH (e.g., greater than about 12). A pharmaceutical composition may comprise, in some embodiments, a base selected from sodium hydroxide, potassium hydroxide, barium hydroxide, strontium hydroxide, rubidium hydroxide, magnesium hydroxide, and combinations thereof. According to some embodiments, an abnormal tissue may be selected from bone cancer, breast cancer, colon cancer, lung cancer, prostate cancer, skin cancer, and combinations thereof. An abnormal tissue may include, in some embodiments, metastatic disease. A method for locoregional treatment of abnormal tissue may include administering, a volume of a pharmaceutical composition (e.g., comprising a base) of less than about two microliters per cubic centimeter of abnormal tissue in some embodiments. A method for locoregional treatment of abnormal tissue may include, according to some embodiments, administering, a volume of a pharmaceutical composition of at least about two microliter per cubic centimeter of abnormal tissue. For example, the volume of the administered composition may be from about two microliters per cubic centimeter of abnormal tissue to about ten microliters per cubic centimeter of abnormal tissue. In some embodiments, a pharmaceutical composition may have a pH greater than about 13.0 and/or greater than about 14.0. A pharmaceutical composition may comprise a salt (e.g., a pharmaceutically acceptable saline), in some embodiments. A pharmaceutical composition may consist of, according to some embodiments, water, a buffer, and sodium hydroxide. A pharmaceutical composition may consists of water, a buffer, a pharmaceutically acceptable carrier, and sodium hydroxide in some embodiments.

### BRIEF DESCRIPTION OF THE DRAWINGS

Some embodiments of the disclosure may be understood by referring, in part, to the present disclosure and the accompanying drawings, wherein:

FIG. 1A illustrates localization of a radiopharmaceutical composition according to a specific example embodiment of the disclosure through a whole-body anterior gamma camera image of a subject (No. 1) taken 120 minutes after administration of a radiopharmaceutical having a pH of 13 and comprising ¹⁶⁶Ho and ¹⁷⁷Lu; and

FIG. 1B illustrates localization of a radiopharmaceutical composition according to a specific example embodiment of the disclosure through a whole-body posterior gamma camera image of a subject (No. 1) taken 120 minutes after administration of a radiopharmaceutical having a pH of 13 and comprising ¹⁶⁶Ho and ¹⁷⁷Lu.

### DETAILED DESCRIPTION

Staging is the process of describing the extent or spread of the disease at the time of diagnosis. Staging is used in determining the choice of therapy and in assessing prognosis. A cancer's stage is based on the primary tumor's size and location in the body and whether it has spread to other areas of the body. There are a variety of staging systems used to classify tumors, varying with the type of cancer. The TNM staging system assesses tumors based on the extent of the primary tumor (T), presence or absence of regional lymph node involvement (N), and the presence or absence of distant metastases (M). With this information, the stage of I, II, III, or IV is assigned (ascending order of severity). The 5 year survival correlates with the stage at the time of diagnosis. For malignant melanoma the five year survival for patients with stage IV disease is approximately 20% at the time of diagnosis.

In some embodiments, cancer treatments may vary depending on the type of cancer as well as the stage. For example, early stage tumors may be treated surgically if the clinical condition of the patient allows it. Early stage melanomas may be cured by surgery alone.

Late stage tumors may be treated non-operatively with chemotherapy, radiation therapy, or both. Some tumors, for example multiple myeloma, lymphoma and seminoma, are very sensitive to radiation therapy, and, in some embodiments, focal lesions may be treated effectively with radiation therapy alone. Radiation may also be used for palliation of pain in late stage tumors. Chemotherapy is used to treat tumors with remote metastases. Chemotherapy may be given as monotherapy or, if desired or required, as combinations of drugs to maximize the effectiveness.

Despite effective chemotherapy, tumors are frequently heterogeneous at the cellular level, leading to mutations that may escape the effectiveness of a particular combination of agents. This is one mechanism leading to relapse, often with a more aggressive progression of disease. The same combination of agents that was effective earlier in the course of a patient's treatment may be ineffective in a relapse. The combination of relapse, lack of response to previous agents, and more aggressive disease lead to a grim prognosis in patients with advanced disease, and few treatment options. This is particularly true of metastatic malignant melanoma and widely disseminated carcinoma.

Toxic side-effects of chemotherapy are common and often lead to significantly reduced quality of life for the patient. Thus, a need has arisen for more targeted therapies that are more selective to the tumor and less toxic to the patient.

The present disclosure relates, according to some embodiments, to methods for treating an abnormal tissue comprising administering to a subject a basic composition. A composition may comprise, for example, a radiopharmaceutical composition comprising at least one unsealed, non-colloidal, radioactive, free metal and/or metal ion moiety. In some embodiments, intratumoral injection of a composition (e.g., a radiopharmaceutical composition) may be used to treat surgically unresectable metastatic disease.

Intratumoral injection with radioisotopes may be used in both animal models and in humans with stage IV malignancies. A composition (e.g., a liquid solution) may be localized to an abnormal tissue (e.g., a tumor) by appropriately adjusting the pH of the composition in some embodiments. For example, an intratumorally injected composition having a high pH may remain at the injection site without requiring glass microspheres, antibodies, or other targeting moieties. According to some embodiments, a composition comprising Ho-166 and Lu-177 may be localized to the region of injection and/or may be effective in treating cutaneous metastases (e.g., nude mouse model with flank tumors injected with the isotopes).

### Compositions

The present disclosure, according to some embodiments, relates to radiopharmaceutical compositions comprising a radionuclide and a solvent (e.g., water). In some embodiments, a radiopharmaceutical composition may comprise only one non-colloidal, radioactive, free metal and/or metal ion moiety. For example, a radiopharmaceutical composition may comprise a non-colloidal, radioactive, free metal and/or metal ion of actinium-225 (²²⁵Ac) antimony-126 (¹²⁶Sb), antimony-127 (¹²⁷Sb), bismuth-212 (²¹²Bi), bismuth-213 (²¹³Bi), cesium-132 (¹³²Cs), europium-136 (¹³⁶Eu), gadolinium-159 (¹⁵⁹Gd), lead-212 (²¹²Pb), lutetium-177 (¹⁷⁷Lu), phosphorous-32 (³²P), promethium-148 (¹⁴⁸Pm), promethium-149 (¹⁴⁹Pm), rubidium-86 (⁸⁶Rb), strontium-89 (⁸⁹Sr), technetium-99m (^{99m}Tc), tin-125 (¹²⁵Sn), or ytterbium-175 (¹⁷⁵Yb). A free metal and/or metal ion or atom may be, according to some embodiments, free of covalent bonds to another molecule and/or unbound by a chelator. According to some embodiments, a radiopharmaceutical composition may comprise a free halide salt comprising, for example, iodine-123 (¹²³I).

According to some embodiments, a radiopharmaceutical may comprise a combination of holmium-166 (¹⁶⁶Ho) and lutetium-177 (¹⁷⁷Lu). For example, a radiopharmaceutical composition may comprise an admixture of a first composition comprising a non-colloidal, radioactive, free metal and/or metal ion of ¹⁶⁶Ho and a second composition comprising a non-colloidal, radioactive, free metal and/or metal ion of ¹⁷⁷Lu. A radiopharmaceutical may comprise, in some embodiments, two radionuclides at any ratio (e.g., weight ratio, molar ratio, and radioactive decay ratio) desired or required. For example, the ratio (whether weight ratio, molar ratio, or radioactive decay ratio) of ¹⁶⁶Ho to ¹⁷⁷Lu may be from about 20:1 to about 5:1, from about 10:1 to about 2:1, from about 5:1 to about 1:1, or from about 2:1 to about 1:5.

A composition may have, according to some embodiments, a pH as desired for the target tissue and/or the stability of one or more components of the composition. A pharmaceutical composition may be formulated to have a basic pH (e.g., pH≧7.0). For example, a composition may have a pH greater than 7.0, greater than about 8.0, greater than about 8.5, greater than about 9.0, greater than about 9.5, greater than about 10.0, greater than about 10.5, greater than about 11.0, greater than about 11.5, greater than about 10.0, greater than about 10.5, greater than about 12.0, greater than about 12.5, greater than about 13.0, greater than about 13.5, and/or up to about 14.0. According to some embodiments, the pH of a composition may be adjusted as desired, for example, using an acid (e.g., HCl, H₂SO₄), a base (e.g., NaOH), and/or a buffer (e.g., phosphate, TRIS, HEPES).

In some embodiments of the disclosure, a composition may have the form of a solution (e.g., an injectable solution, a liquid solution, a syrup, a thick syrup or a paste), a jelly or a mucilage. A jelly may comprise, according to some embodiments, a thickening agent (e.g., a water soluble polysaccharide and/or a synthetic cellulose derivative which swells in water). Non-limiting examples of a thickening agent for a jelly may include acacia, chondrus, gelatin, xanthan gum carboxymethylcellulose, hydroxyethylcellulose, hydroxypropylcellulose, hydroxypropylmethylcellulose, and/or chitosan. A mucilage may be a thick, viscid, adhesive liquid. A mucilige may be produced by dispersing or dissolving gum in water, or by extracting mucilaginous principles from vegetable substances with water. Non-limiting examples of a mucilage include, for example, acacia mucilage and tragacanth mucilage. In some embodiments, a mucilage may comprise a thickening agent (e.g., a water soluble polysaccharide and/or a synthetic cellulose derivative which swells in water). According to some embodiments, chitosan may thicken under some conditions including, for example, a high pH of the injected mixture. Under some conditions, chitosan may become a gel at high pH (13), which may limit spread of the isotope.

A composition may be administered, according to some embodiments, with or in a pharmaceutically-acceptable additive (e.g., carriers, excipients, and diluents). Suitable carriers include buffers such as phosphoric acid, citric acid and other organic acids; antioxidants such as ascorbic acid; low-molecular weight polypeptides; proteins such as serum albumin, gelatin and immunoglobulin; hydrophilic polymers such as polyvinylpyrrolidone; amino acids such as glycine, glutamine, arginine or lysine; monosaccharides such as mannose or dextrin, disaccharides, other carbohydrates; metal ions such as zinc, cobalt or copper; sugar alcohols such as mannitol or sorbitol; salt-forming counter ions such as sodium; and/or non-ionic surfactants such as polysorbate, PLURONIC (block copolymers based on ethylene oxide and propylene oxide) or polyethylene glycol (PEG). Excipients and diluents may be selected from the group consisting of magnesium stearate, calcium carbonate, starch-gelatin paste, talc, aluminum salt, phenoxyethyl ethanol, water, physiological salt solution, lactose, dextrose, sucrose, sorbitol, mannitol, calcium silicate, cellulose, methyl cellulose, amorphous cellulose, polyvinylpyrolidone, methylhydroxy benzoate, propylhydroxybezoate, and a mineral oil. Other optional components, e.g., stabilizers, buffers, preservatives, flavorings, chelators and the like, may be added.

### Methods of Therapy

The present disclosure, according to some embodiments, relates to methods for treating an abnormal tissue in a subject. For example, methods and/or compositions of the present disclosure may be used to treat malignant or benign neoplasms, including solid tumors. Non-limiting examples of conditions that may be treated using this invention include: (a) abdominal neoplasms; (b) bone neoplasms; (c) breast neoplasms; (d) digestive system neoplasms; (e) endocrine gland neoplasms; (f) eye neoplasms; (g) brain, head and neck neoplasms; (h) hematologic neoplasms; (i) nervous system neoplasms; (j) pelvic neoplasms; (k) skin neoplasms; (l) soft tissue neoplasms; (m) splenic neoplasms; (n) thoracic neoplasms; (o) urogenital neoplasms; (p) and additional cancers including renal carcinoma, lung cancer, melanoma, Barrett's esophagus, and metaplasia pre-cancer cells. Metastatic malignant melanoma and widely disseminated metastatic carcinoma, which may be associated with a grave prognosis, may be treated according to some embodiments of the disclosure. Without limiting any specific embodiment to any particular mechanism of action, treatment may include promoting or creating conditions which (a) slow or arrest cell proliferation of the abnormal tissue and/or (b) lead to the death of one or more cells of the abnormal tissue.

In some embodiments, a method may include administering a radiopharmaceutical to a subject (e.g., a human and/or a non-human animal). Non-limiting examples of non-human subjects may include non-human primates (e.g., gorillas, monkeys, marmosets), horses, dogs, cats, cattle, pigs, sheep, birds, and/or ferrets.

According to some embodiments, a method may comprise administering a composition by injection or through any other effective mode. For example, a composition may be delivered to the subject by injection, surgical implantation, procedures similar to those used for needle biopsies, cannulation, or any other useful mode known in the art. In some embodiments, the volume of a composition to be administered may be determined with due consideration for the size and health of the recipient as well as the size, condition, and location of the target tissue within the recipient. The volume of administered composition may be from about one tenth of a microliter (0.1 μL) to about ten milliliters (10 mL), from about one microliter (1 μL) to about ten microliters (10), from about ten microliters (10 μL) to about one milliliter (1 mL), from about one milliliter (1 mL) to about ten milliliters (10 mL), and/or over about ten milliliters (10 mL).

For any particular subject, specific dosage regimens (e.g., specific activity, isotope concentration, isotope ratio (if more than one), volume, and scheduling for each administration) may be adjusted, in some embodiments, over time according to the individual need and the judgment of the user (e.g., an attending medical professional or person supervising administration) in light of the subject's health, the nature of the target abnormal tissue, and/or any other relevant factors. For example, dosing of a composition may depend, at least in part, on absorption, inactivation, and/or excretion rates of a composition and/or its active agent(s) as well as other factors known to those of skill in the art. Dosage values may vary with the severity of the condition to be alleviated. For example, repeated administrations and/or larger volumes may be administered to larger tumors (e.g., five microliters of a composition comprising 6 mCi ¹⁶⁶Ho and 3 mCi ¹⁷⁷Lu per cubic centimeter of tumor). A method may comprise, according to some embodiments, administering a radiopharmaceutical as many times as desired and/or needed (e.g., to deliver a therapeutically effective dose). For example, a radiopharmaceutical composition may be delivered to a subject in a single administration or two or more sequential administrations.

A radiopharmaceutical composition may remain, according to some embodiments, at the site of administration (e.g., injection) for a period of time sufficient to contact the abnormal tissue with a therapeutically effective amount of radiation (e.g., alpha, beta, gamma). For example, at least a majority (e.g., at least about 80%, at least about 90%, and/or at least about 95%) of the remaining isotope (i.e., still present after radioactive decay) may be physically located within one or two centimeters of an administration site for up to about 3 hours, up to about 6 hours, up to about 12 hours, up to about 24 hours, up to about 2 days or over about two days.

Successful use of a composition in a subject may be determined using one or more possible metrics known in the art, including but not limited to, a reduction in solid tumor size, a decreased level of tumor markers, an alleviation of symptoms, and/or an inability to detect the neoplasm using one or more of the diagnostic techniques known in the art.

As will be understood by those skilled in the art who have the benefit of the instant disclosure, other equivalent or alternative compositions, devices, methods, and systems for treating an abnormal tissue can be envisioned without departing from the description contained herein. Accordingly, the manner of carrying out the disclosure as shown and described is to be construed as illustrative only.

Persons skilled in the art may make various changes in the formulation of a radiopharmaceutical composition without departing from the scope of the instant disclosure. For example, the pH, volume, and/or constituents (e.g., kind, concentration, specific activity, ratio) may be varied. In addition, the volume, dose, and/or number of administrations may be scaled up (e.g., to be used for larger tumors, more numerous tumors, and/or adult subjects) or down (e.g., to be used for smaller tumors, fewer tumors, and/or juvenile subjects) to suit the needs and/or desires of a practitioner.

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, a range of endpoint of about 50 may one the one hand include 50.5, but not 52.5 or 55 in the context of a range of about 5 to about 50 and, on the other hand, include 55, but not 60 or 75 in the context of a range of about 0.5 to about 50. In addition, it may be desirable, in some embodiments, to mix and match range endpoints. Also, in some embodiments, each figure disclosed (e.g., in one or more of the Examples and/or Drawings) may form the basis of a range (e.g., +/−about 10%, +/−about 100%) and/or a range endpoint. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein. As shown in Example 2 and Example 3, Ho-166 and Lu-177 remained within the area of injection when injected intratumorally in a basic solution. These studies have also shown a decrease in the size of cutaneous metastases. The study presented in Example 4 is designed to confirm the observations of decreased tumor size related to intratumoral injection of the radioisotopes.

### Example 1

### Pre-Clinical Study of a ²¹³Bi Radiopharmaceutical

Preclinical studies of intratumoral injection of Bi-213 have utilized a targeting monoclonal antibody 9.2.27 and a mouse model. The Bi-213 was chelated to cDTPA which was covalently linked to the monoclonal antibody in these studies. Although the alpha particle-producing immunoconjugate resulted in a short term weight loss in the nude mice at high doses, no such weight loss was observed when lower doses of radiation were injected. The tolerance dose value for an intratumoral injection using this construct was approximated to be 10 mCi/kg weight.

### Example 2

### Pre-Clinical Study of a ¹⁶⁶Ho+¹⁷⁷Lu Radiopharmaceutical

Preclinical studies have utilized intratumoral injection of both Ho-166 and Lu-177 with multiple chelators as well as multiple pH formulations. The results indicated that a biodistribution similar to that obtained with localizing antibodies (Example 1) may be obtained with a formulation at appropriate pH. For example, preclinical studies in rats and dogs have shown that directly injected formulations of Ho-166 and Lu-177 in the presence of a strong base remain in the region of the injection. Given the longer half-life of Lu-177, it has been possible to demonstrate that the mixture remains localized in the region of injection for weeks.

Ho-166 has been used with a polymer of 2-deoxy-2-amino-D-glucose (chitosan) to deliver radiation to hepatocellular carcinomas. The formulation of Ho-166/Lu-177 used in this protocol similarly remains in the region of injection.

### Example 3

### Clinical Study of Intratumoral Injection of a Radiopharmaceutical

A clinical study utilizing a radiopharmaceutical comprising Ho-166 and Lu-177 injected into cutaneous metastases of human subjects has shown that the radioisotopes remain in the region of injection.

Isotope. Vials of Lu-177 and Ho-166 are received in 0.1 N HCl. An example radiopharmaceutical composition was prepared by (a) transferring 3 mCi of Lu-166 and 6 mCi of Ho-166 into a V-shaped sterile vial using a micropipette (1-5 microliters of each), (b) adding the same number of microliters of 1.0 N NaOH to the vial as the sum of volume added of Ho-166 and Lu-177, and (c) adding enough sterile water to make a total of 15 μL of solution. An aliquot (e.g., 5 μL) of the composition may be taken up into a syringe and calibrated. The calibrated aliquot was used for injection into a subject. An aliquot was spotted onto pH paper and determined to have a pH of 13.

Administration. Each subject received a test composition. Cutaneous metastases were injected utilizing a 10 μL or 25 μL chromatographic syringe and needle. All tumors were injected with 5 μL of solution containing about 1 mCi ¹⁷⁷Lu and 2 mCi ¹⁶⁶Ho. Radioisotopes were received in acidic solution (HCl) and the pH was adjusted using sodium hydroxide. Therefore, the final composition included some sodium chloride.

The primary objective of this example study was to determine whether an intratumorally injected radiopharmaceutical (Ho-166 and Lu-177) would remain at or near the site of injection. Using a gamma camera, the localization of the injected isotopes may be determined up to 30 days after injection. Ho-166 has a half-life of 26.76 hours and Lu-177 has a half-life of 6.73 days. Therefore, at day 30, less than 0.20% of the radioactive Ho-166 and less than 6.25% of the radioactive Lu-177 is expected to remain in the subject.

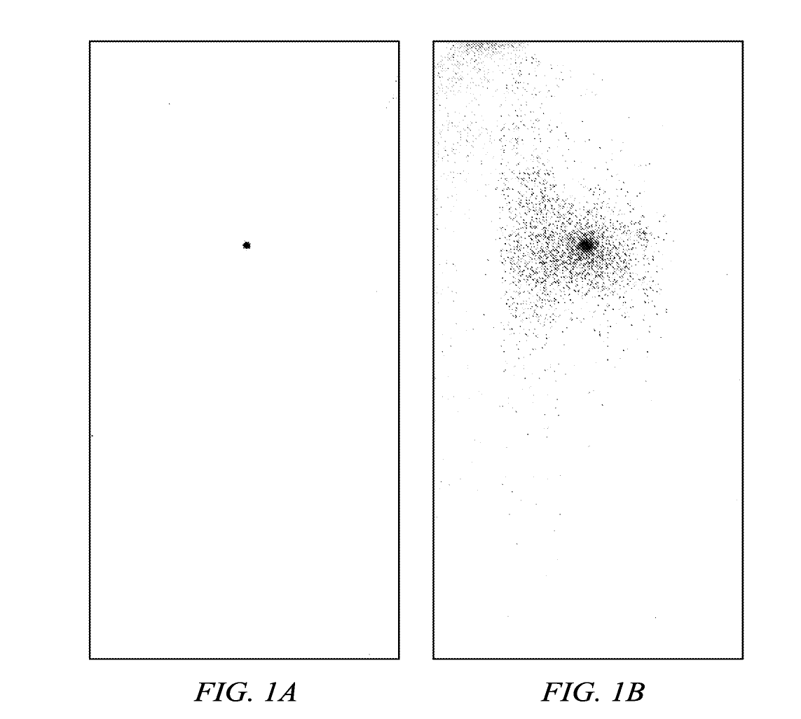

FIG. 1A and FIG. 1B, which are whole body scans of patient number 1 taken 120 minutes after injection, are representative of scans taken of all subjects in this trial. The cutaneous metastasis, which was injected in patient No. 1, was located just below the inferior rib in the midclavicular line on the left side. The dark spot in the anterior image (FIG. 1A) is approximately 1 cm in diameter and corresponds to the injection site. The dark spot in the posterior gamma camera image (FIG. 1B) is larger in apparent size (about 2 cm) due to the anterior location of the tumor injected and the distance of that tumor from the posterior detector.

As shown in Table 1 below, laboratory data collected during the trial indicated that administration of the radiopharmaceuticals did not adversely effect bone marrow functions (hemoglobin and white blood cells), renal function (creatinine) or hepatic function (aspartate aminotransferase). throughout the radioactive Lu-177 is expected to remain in the subject. The changes observed were attributable to the general the medical condition of the patients.

TABLE 1
Clinical Laboratory Results

| | Hemoglobin Level | | | White Blood Cell Count | | |
|---|---|---|---|---|---|---|
| Patient | Day 1 | Day 8 | Day 30 | Day 1 | Day 8 | Day 30 |
| 1 | 12.30 | 12.40 | 13.00 | 8650 | 8910 | 8600 |
| 2 | 13.00 | 13.70 | 13.40 | 5370 | 5580 | 4910 |
| 3 | 11.10 | 11.20 | 9.00 | 7310 | 5960 | 4600 |
| 4 | 14.90 | 15.60 | | 11170 | 14410 | |
| 5 | 12.00 | 11.80 | 11.40 | 3190 | 3550 | 4880 |
| 6 | 10.60 | 8.60 | 12.40 | 6540 | 590 | 7170 |
| | Aspartate | | | | | |
| | Aminotransferase | | | Creatinine | | |
| | (Normal: Minimum | | | (Normal: Minimum | | |
| | of 48 mg/dl) | | | of 1.3 mg/dl) | | |
| Patient | Day 1 | Day 8 | Day 30 | Day 1 | Day 8 | Day 30 |
| 1 | 75 | 72 | 74 | 0.5 | 0.6 | 0.7 |
| 2 | | 110 | 103 | 0.7 | 0.6 | 0.7 |
| 3 | 146 | 235 | 48 | 0.7 | 1.0 | 0.9 |
| 4 | 66 | 151 | | 0.8 | 1.0 | |
| 5 | 40 | 35 | 37 | 1.0 | 1.0 | 1.0 |
| 6 | 36 | 34 | 71 | 0.7 | 0.7 | 0.6 |

A secondary objective of this example study was to evaluate the potential of an intratumorally injected radiopharmaceutical (Ho-166 and Lu-177) to reduce the volume of cutaneous metastases of melanoma or scattered metastasis of carcinoma. All six patients included in the study presented/displayed metastatic carcinoma of the breast. Reductions the size in all six tumors were observed as shown in Table 2 below.

TABLE 2
Reduction of Tumor Size

| Patient | Day 0 | Day 8 | Day 15 | Day 22 | Day 30 |
|---|---|---|---|---|---|
| 1 | 225 mm² | 195 mm² | | 121 mm² | 72 mm² |
| 2 | 224 mm² | 120 mm² | | 90 mm² | 0 mm² |
| 3 | 640 mm² | 520 mm² | 300 mm² | 260 mm² | 676 mm² |
| 4 | 400 mm² | 323 mm² | 0 mm² | 0 mm² | |
| 5 | 225 mm² | 156 mm² | 100 mm² | 0 mm² | 0 mm² |
| 6 | 54 mm² | 0 mm² | 0 mm² | 0 mm² | 0 mm² |

There was no evidence of toxicity in any of the six patients treated. One patient (patient number 4) died because of the progression of the disease before day 30 of the test. Although favorable results were observed with the treated tumor, the patient had other lesions that were not treated. That patient's hepatic enzymes increased between Day 1 and Day 8, which is consistent with progression of the disease. Patient 4 showed no signs of renal toxicity or bone marrow suppression. Patient 3's cutaneous metastasis was the largest tumor injected and initially displayed a marked reduction in size.

Survey responses from the patients indicate that the patients experienced no reduction in their quality of life attributable to the intratumoral injections. The patients did report a positive emotional response upon seeing the size of their tumor decrease.

### Example 4

### Clinical Study of Intratumoral Injection of a Radiopharmaceutical

This example describes a single center, open-label, single dose study to evaluate the safety of intratumoral injection of Ho-166 and Lu-177 in subjects with advanced malignant melanoma or metastatic carcinoma refractory to standard therapies.

Subjects (n=20) may include men and non-pregnant, non-lactating women 18 years of age or older who have Stage 4 relapsed/refractory malignant melanoma or extensively metastatic carcinoma. For example, subjects selected for administration may (a) have a pathologically proven diagnosis of malignant melanoma or metastatic carcinoma, refractory to standard therapies, (b) be able to undergo imaging procedures including radioisotopic scans, and (c) be able to give informed consent for the study procedure. A candidate subject who otherwise may benefit from administration of a composition of the disclosure, may be excluded from a particular experimental protocol, if desired. For example, a subject may be excluded if the subject (a) has concurrent medical conditions that in the judgment of the treating physician may affect the subject's ability to complete the planned study, (b) is pregnant and/or lactating, (c) has a myelodysplastic syndrome, aplastic anemia, anemia or neutropenia due to diminished bone marrow reserve, (d) has a renal insufficiency and is not stable on medical management or dialysis, or (e) has or has had liver failure.

Preparation. Each subject may have a laboratory assessment/evaluation of major organ function, hematology, serum chemistry, and urinalysis within 48 hrs prior to intratumoral injection of a radioisotope. Each subject may also complete a medical history, a physical examination, and/or a quality of life survey. Prior to administration of a composition, tumor size may be measured.

Isotope. Vials of Lu-177 and Ho-166 are received in 0.1 N HCl. An example radiopharmaceutical composition may be prepared by (a) transferring 3 mCi of Lu-166 and 6 mCi of Ho-166 into a V-shaped sterile vial using a micropipette (1-5 microliters of each), (b) adding the same number of microliters of 1.0 N NaOH to the vial as the sum of volume added of Ho-166 and Lu-177, and (c) adding enough sterile water to make a total of 15 μL of solution. An aliquot (e.g., 5 μL) of the composition may be taken up into a syringe and calibrated. The calibrated aliquot may be used for injection into a subject.

Administration. Each subject may receive a control composition or a test composition. Cutaneous metastases may be injected utilizing a 10 μL or 25 μL chromatographic syringe and needle. Tumors smaller than 2.0 cm will be injected at the center of the tumor mass. Tumors larger than 2.0 cm will be injected in two locations approximately one third of the diameter from the edge of the tumor along the major axis of the tumor. If a patient has more than one cutaneous metastasis, then one of the tumors will be injected with the 1N sodium hydroxide solution used to prepare the radioisotopes for intratumoral injection.

Monitoring. Each subject may undergo whole body anterior and posterior planar imaging at 15, 120 minutes post injection of the radioisotopes and on day 8 to evaluate the biodistribution to whole body, critical organs, and tumors. Tumor size may be measured on days 8 and/or 30. Adverse events (AEs) may be systematically elicited and recorded without leading questions, at each follow-up visit. Outpatient follow up visits may occur at 30 days±2 days after injection of the tumor. History, safety labs, and assessment of adverse events may be obtained.

Safety laboratory tests may be evaluated on study days 8 and 30. Quality of Life surveys may be taken on study Day 30. Subject response may be graded, if possible, as minimal response (“MR”), partial response (“PR”), complete response (“CR”), no response (“NR”), stable disease (“SD”) or progressive disease (“PD”) using clinical evaluation and laboratory values as the primary response criteria. Subjects may be monitored for adverse events from the time of consent and registration to 60 days post-tumor injection.

Table 3 below shows a flow diagram of the study.

TABLE 3
Study Flow Diagram

| Day | Procedure |
|---|---|
| −1 or 0 | Safety Lab Studies |
| | Physical Examination |
| | Medical History |
| | Quality of Life |
| 1 | Measure Tumor |
| | Inject Composition (e.g., ¹⁶⁶Ho + ¹⁷⁷Lu) |
| 1 (+15 minutes) | Image patient |
| 1 (+120 minutes) | Image patient |
| 8 | Safety Lab Studies |
| | Measure Tumor |
| | Image patient |
| 30 ± 2 | Safety Lab Studies |
| | Physical examination |
| | Measure Tumor |
| | Quality of Life |

What is claimed is:

1. A method for locoregional treatment of abnormal tissue in a human subject, the method comprising:
administering to a subject in the region of the abnormal tissue a radiopharmaceutical composition having a pH greater than about 9.0 and comprising an admixture of:
a first radionuclide composition comprising an unsealed, non-colloidal, radioactive, free holmium-166 (¹⁶⁶Ho) and/or unsealed, non-colloidal, radioactive, free metal ion of holmium-166 (¹⁶⁶Ho); and
a second radionuclide composition comprising an unsealed, non-colloidal, radioactive, free lutetium-177 (¹⁷⁷Lu) and/or an unsealed, non-colloidal, radioactive, free metal ion of lutetium-177 (¹⁷⁷Lu).

2. A method according to claim 1, wherein the free metal ion of holmium-166 is ¹⁶⁶Ho³⁺ and the second radioactive free metal ion of lutetium-177 is ¹⁷⁷Lu³⁺.

3. A method according to claim 1, wherein the abnormal tissue is selected from the group consisting of bone cancer, breast cancer, colon cancer, lung cancer, prostate cancer, skin cancer, and combinations thereof.

4. A method according to claim 1, wherein the volume of the administered composition is less than about two microliters per cubic centimeter of abnormal tissue.

5. A method according to claim 1, wherein the volume of the administered composition is at least about two microliter per cubic centimeter of abnormal tissue.

6. A method according to claim 1, wherein the volume of the administered composition is from about two microliters per cubic centimeter of abnormal tissue to about ten microliters per cubic centimeter of abnormal tissue.

7. A method according to claim 1, wherein the radiopharmaceutical composition has pH greater than about 10.0.

8. A method according to claim 1, wherein the radiopharmaceutical composition has pH greater than about 11.0.

9. A method according to claim 1, wherein the radiopharmaceutical composition has pH greater than about 12.0.

10. A method according to claim 1, wherein the radiopharmaceutical composition has pH up to about 14.0.

11. A method according to claim 1, wherein the radiopharmaceutical composition further comprises a salt.

12. A method according to claim 1, wherein the radiopharmaceutical composition further comprises a base selected from the group consisting of sodium hydroxide, potassium hydroxide, barium hydroxide, strontium hydroxide, rubidium hydroxide, magnesium hydroxide, and combinations thereof.

13. A method according to claim 1, wherein at least eighty percent of the total remaining radioactivity of (i) the first radionuclide composition or (ii) the second radionuclide composition remains within one centimeter of the injection site for at least two hours after the administering.

14. A method according to claim 1, wherein at least eighty percent of the total remaining radioactivity of (i) the first radionuclide composition or (ii) the second radionuclide composition remains within one centimeter of the injection site for at least one half life of the radionuclide composition with the shorter half life after the administering.

15. A method according to claim 1, wherein less than twenty percent of the total remaining radioactivity of (i) the first radionuclide composition or (ii) the second radionuclide composition is located more than one centimeter away from the injection site within the first twelve hours after the administering.

16. A method according to claim 1, wherein less than twenty percent of the total remaining radioactivity of (i) the first radionuclide composition or (ii) the second radionuclide composition is located more than one centimeter from the injection site within a time after the administering that is equivalent to one half life of the radioactive free metal ion with the shorter half life.

17. A method according to claim 1, wherein the administering comprises injecting the radiopharmaceutical composition.

18. A method according to claim 17, wherein the injecting the radiopharmaceutical composition comprises intratumorally injecting the radiopharmaceutical composition, intralesionally injecting the radiopharmaceutical composition, intramuscularly injecting the radiopharmaceutical composition, interstitially injecting the radiopharmaceutical composition, intrathecally injecting the radiopharmaceutical composition, intraosseously injecting the radiopharmaceutical composition, intraorbitally injecting the radiopharmaceutical composition, intraparenchymally injecting the radiopharmaceutical composition, or combinations thereof.

19. A method according to claim 1, wherein the administering comprises injecting the radiopharmaceutical composition into or through a bone.

20. A method according to claim 19 further comprising drilling a hole in the bone.

21. A method for the locoregional treatment of abnormal tissue, the method comprising:
administering to a subject in the region of the abnormal tissue a radiopharmaceutical composition having a pH greater than about 9.0 and comprising an unsealed, non-colloidal, radioactive, free metal and/or metal ion, wherein the metal and/or metal ion is selected from the group consisting of antimony-126 (¹²⁶Sb) antimony-127 (¹²⁷Sb), cesium-132 (¹³²Cs), europium-136 (¹³⁶Eu), (gadolinium-159 (¹⁵⁹Gd), lutetium-177 (¹⁷⁷Lu), phosphorous-32 (³²P), promethium-148 (¹⁴⁸Pm), promethium-149(¹⁴⁹Pm), rubidium-86 (⁸⁶Rb), strontium-89 (⁸⁹Sr), tin-125 (¹²⁵Sn), ytterbium-175 (¹⁷⁵Yb), and ions thereof.

22. A method for the locoregional treatment of abnormal tissue, the method comprising:
administering to a subject in the region of the abnormal tissue a radionuclide-free pharmaceutical composition having a pH greater than about 12.0 and comprising a base selected from the group consisting of sodium hydroxide, potassium hydroxide, barium hydroxide, strontium hydroxide, rubidium hydroxide, magnesium hydroxide, and combinations thereof.

23. A method according to claim 22, wherein the subject is human.

24. A method according to claim 22, wherein the subject is a non-human animal.

25. A method according to claim 22, wherein the abnormal tissue is selected from the group consisting of bone cancer, breast cancer, colon cancer, lung cancer, prostate cancer, skin cancer, and combinations thereof.

26. A method according to claim 22, wherein the volume of the administered composition is less than about two microliters per cubic centimeter of abnormal tissue.

27. A method according to claim 22, wherein the volume of the administered composition is at least about two microliter per cubic centimeter of abnormal tissue.

28. A method according to claim 22, wherein the volume of the administered composition is from about two microliters per cubic centimeter of abnormal tissue to about ten microliters per cubic centimeter of abnormal tissue.

29. A method according to claim 22, wherein the pharmaceutical composition has pH greater than about 13.0.

30. A method according to claim 22, wherein the pharmaceutical composition has pH up to about 14.0.

31. A method according to claim 22, wherein the pharmaceutical composition further comprises a salt.

32. A method according to claim 22, wherein the pharmaceutical composition consists of water, a buffer, and the sodium hydroxide.

33. A method according to claim 22, wherein the pharmaceutical composition consists of water, a buffer, a pharmaceutically acceptable carrier, and the sodium hydroxide.
